(19) United States
(12) Patent Application Publication
(10) Pub. No.: US 2026/0263024 A1
(43) Pub. Date: Sep. 10, 2026

(54) MOVING FOCAL SPOT TUBE CALIBRATION

(71) Applicant: Hologic, Inc., Marlborough, MA (US)

(72) Inventors: Baorui REN, Andover, MA (US); David AIZER, Danbury, CT (US); Tushita PATEL, Danbury, CT (US); Jeffrey PAIGE, Wallkill, NY (US); Richard AMOS, Wilmington, DE (US)

(73) Assignee: Hologic, Inc., Marlborough, MA (US)

(21) Appl. No.: 19/166,326

(22) PCT Filed: Mar. 22, 2024
(86) PCT No.: PCT/US2024/021213

§ 371 Date: Sep. 17, 2025

Related U.S. Application Data
(60) Provisional application No. 63/491,773, filed on Mar. 23, 2023.

(51) Int. Cl. A61B 6/58; A61B 6/02; A61B 6/40

(52) U.S. Cl. CPC A61B 6/583; A61B 6/025; A61B 6/4021

(57) ABSTRACT

System and method for determining a predetermined ramp value for an x-ray tube using a sharpness test object positioned between the x-ray tube and an x-ray detector by applying a plurality of ramp values to the x-ray tube; for each applied ramp value, emitting an x-ray energy from the x-ray tube so as to obtain an x-ray image; calculating a figure of merit (FOM) to quantify a motion blur in each x-ray image; and identifying a highest FOM, wherein the highest FOM corresponds to the predetermined ramp value.

### CROSS-REFERENCE TO RELATED APPLICATIONS

This application is being filed as a PCT International Patent Application and claims priority to U.S. Provisional Patent Application No. 63/491,773, filed Mar. 23, 2023, entitled “MOVING FOCAL SPOT TUBE CALIBRATION,” which is incorporated herein by reference in its entirety.

### BACKGROUND

Breast tomosynthesis is an imaging technology in that images of a stationary compressed or immobilized breast are acquired at multiple angles during an imaging scan. The images are organized as a series of thin high-resolution image “slices” that can be displayed individually, in a dynamic ciné mode, or as a synthesized image. Breast tomosynthesis systems move the x-ray source to a variety of different imaging positions relative to an x-ray detector during image acquisition. Reconstructed tomosynthesis slices advantageously reduce or eliminate problems caused by tissue overlap and structure noise in two-dimensional mammography imaging. However, movement of the x-ray source introduces some technological complications.

Typical tomosynthesis systems are arranged to either continuously traverse a path during an image scan or utilize stop-and-start scanning procedures. The x-ray source is activated for an exposure time of about 10 ms to 100 ms as the x-ray source moves into each of several imaging locations in the imaging path, and exposure is repeated with a cycle period of 200 ms to 2 seconds. After each exposure the x-ray source is deactivated. As the x-ray source moves between imaging locations, the contents of the digital image detector are read out and stored. There is a minimum time period associated with reading the image from the digital detector, and the overall speed of the tomosynthesis scan is determined by the minimum time period for detector read, the exposure time at each location, and the number of exposures. For continuous scans, the x-ray source is moved through space during each exposure period in a tomosynthesis system, that may result in blurring that may reduce diagnostic accuracy.

### SUMMARY

Aspects of the present disclosure relate to a method of determining a predetermined ramp value for an x-ray tube using a sharpness test object positioned between the x-ray tube and an x-ray detector. In some aspects, the method includes applying a plurality of ramp values to the x-ray tube; for each applied ramp value, emitting an x-ray energy from the x-ray tube so as to obtain an x-ray image; calculating a figure of merit (FOM) to quantify a motion blur in each x-ray image; and identifying a highest FOM, wherein the highest FOM corresponds to the predetermined ramp value.

In some embodiments, the ramp value is a current. In other examples, the ramp value is a voltage. In other aspects, the predetermined ramp value determines a displacement of a focal spot on an anode of the x-ray detector during an x-ray exposure. In further examples, the method includes plotting each FOM against the ramp value applied on an FOM-ramp value plot; and determining one or more additional data points on the FOM-ramp value plot to obtain an interpolated FOM-ramp value plot. In still further examples, determining one or more additional data points on the FOM-ramp value plot comprises performing a curve fitting. In yet further aspects, the curve fitting is a polynomial curve fitting.

In other examples presented herein, calculating the FOM comprises measuring a blur length in each x-ray image. In some aspects, calculating the FOM comprises measuring a width of a line spread function for each x-ray image. In some aspects, calculating the FOM comprises measuring an amplitude of a modulation transfer function for each x-ray image. In some implementations, each of the plurality of ramp values applied comprises a different ramp value. In other aspects, the sharpness test object is a modulation transfer function edge phantom.

In some examples, the sharpness test object is a wire phantom. In other aspects, the sharpness test object is point phantom. In still other aspects, emitting the x-ray energy is performed during a tomosynthesis scan sequence. In further examples, the tomosynthesis scan is either in a clockwise direction or a counterclockwise direction. In some implementations, the method is executed at a first voltage station and repeated at a second voltage station. In further examples, the method includes plotting a voltage range graph with a first predetermined ramp value with the first voltage station and a second predetermined ramp value with the second voltage station; and interpolating a third predetermined ramp value at a third voltage station based on the voltage range graph.

Some aspects of the present disclosure relate to a method of mitigating focal spot blur in x-ray images by displacing a focal spot relative to motion of an x-ray tube. The method includes determining a predetermined ramp current by: applying a plurality of ramp values to the x-ray tube; for each applied ramp value, emitting an x-ray energy from the x-ray tube so as to obtain an x-ray image; calculating a figure of merit (FOM) to quantify a motion blur in each x-ray image; and identifying a highest FOM, wherein the highest FOM corresponds to the predetermined ramp value; and applying the predetermined ramp current to the x-ray tube during image capture.

Other aspects of the present disclosure relate to a system including a computer-readable memory storing executable instructions; and one or more processors in communication with the computer-readable memory, wherein, when the one or more processors execute the executable instructions, the one or more processors perform: applying a plurality of ramp values to the x-ray tube; for each applied ramp value, emitting an x-ray energy from the x-ray tube so as to obtain an x-ray image; calculating a figure of merit (FOM) to quantify a motion blur in each x-ray image; and identifying a highest FOM, wherein the highest FOM corresponds to the predetermined ramp value.

A variety of additional inventive aspects will be set forth in the description that follows. The inventive aspects can relate to individual features and to combinations of features. It is to be understood that both the forgoing general description and the following detailed description are exemplary and explanatory only and are not restrictive of the broad inventive concepts upon that the embodiments disclosed herein are based.

### BRIEF DESCRIPTION OF THE DRAWINGS

The accompanying drawings, that are incorporated in and constitute a part of the description, illustrate several aspects of the present disclosure. A brief description of the drawings is as follows:

FIG. 1 depicts an example breast tomosynthesis system.

FIGS. 2 and 3 depict synchronized movement of the static focal spot and x-ray tube.

FIG. 4 depicts an example workflow of calibrating a moving focal spot x-ray tube.

FIG. 5 depicts an example plot of a figure of merit (FOM) to ramp value, using the modulation transfer function (MTF) for the FOM.

FIG. 6 depicts an example graph of ramp value to voltage station, using a supplied current as the ramp value.

FIG. 7 depicts an example of a suitable operating environment in that one or more of the present examples can be implemented.

### DETAILED DESCRIPTION

Performing tomosynthesis imaging using a moving focal spot (MFS) x-ray tube has provided significant advancement in the field of medical imaging as an ultra-fast tomosynthesis scan can be done with nearly zero focal spot motion blur in images. While an MFS x-ray tube offers unique capability to cancel focal spot motion blur, practical control and a refined calibration method is needed to achieve the desired minimal motion blur in a tomosynthesis system. A reliable and efficient calibration method is an important aspect in effective implementation of MFS tube technology.

The present disclosure describes methods and systems for MFS tube calibration allowing users to quickly derive the tube control parameters, or ramp values, needed to effectively configure the MFS tube system. Aspects of the present disclosure further describe methods and systems to perform quality control evaluation tests to monitor the performance of the MFS x-ray tube in the course of operations.

As disclosed herein, during the MFS tube calibration, a calibration scan is run using different ramp values sent to the MFS tube for a number of exposures to produce a different amount of focal spot deflection in each exposure. For example, the calibration scan may be run using a standard tomosynthesis sequence of 15 exposures, and therefore evaluate 15 different ramp values with a different ramp value applied to each of the 15 exposures. Exposures are executed with a cycle period which may be 200 ms to 2 seconds. In some embodiments, the cycle period may be reduced, and in some cases may be, for example, 150 ms, 100 ms, or 80 ms, by reducing the delay time between exposures.

A phantom may be included in the images captured during the calibration scan to provide a measurement reference for determining the sharpness of the images produced. The phantom is a high contrast object oriented within the exposure frame and acts as a sharpness test object. Various shapes and configurations are possible for the sharpness test object, such as a point phantom (e.g., a small spherical object such as a BB), a wire phantom (e.g., a thin wire), or a phantom edge, or multiple combinations of these phantoms.

As discussed in further detail herein, a plurality of phantom images are measured with a different ramp value in each exposure or frame. A blur measurement is taken or an image sharpness calculation is performed, such as by a modulation transfer function (MTF) calculation, along the scan direction. The ramp value associated with a smallest or shortest blur measurement among the images captured is saved as a predetermined ramp value for that applied voltage or voltage station. MTF provides a measure of a sharpness of an image, so a highest MTF value may correspond to a desired predetermined ramp value.

Predetermined ramp values at multiple voltage stations may be determined. In embodiments, a number of voltage stations within a range are measured and values for all other voltage stations in a range are generated through curve fitting and data interpolation. For example, four key voltage stations within a range may be calibrated, e.g., 25 kV, 30 kV, 40 kV, 49 kV, and the calibration procedure determines an optimal ramp value for each voltage station in a range, e.g., from 20 kV to 49 kV, and updates the system accordingly to set the predetermined ramp value.

As used herein, the term “optimal” refers to any ramp value that displays improved performance as compared with default ramp value, a prior predetermined ramp value, other ramp values in a calibration set, etc. For example, an optimal ramp value may refer to a ramp value associated with a lowest blur, among a plurality of ramp values evaluated, when applied to the x-ray tube. An optimal ramp value does not necessarily refer to a best or most favorable ramp value overall, rather, an optimal ramp value may be a most optimal value among an evaluated set based on predetermined criteria, e.g., lowest blur length or highest MTF. A predetermined ramp value may generally be a ramp value that produces an image with a highest measured sharpness or a lowest measured blur. In examples, the predetermined ramp value may be an optimal ramp value, in that no further increases in image sharpness may be possible or necessary by further adjustment of the ramp value, in that the desired improvements have been obtained. In examples, the predetermined ramp value need not be optimal in that a sharper image or an image with further reduced blurring may theoretically be possible.

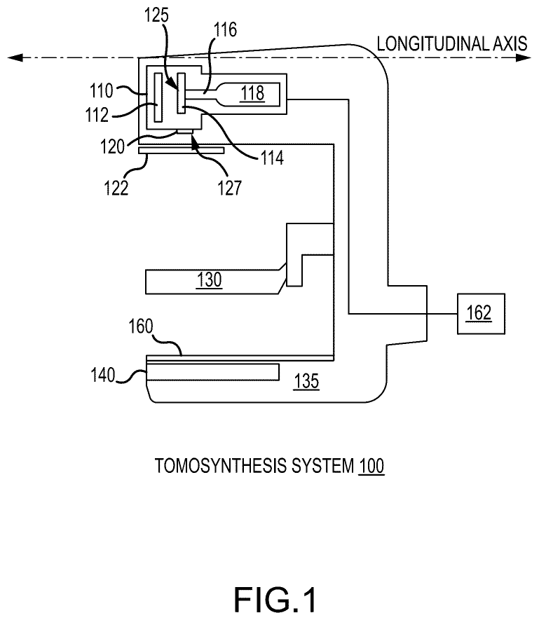

FIG. 1 illustrates a tomosynthesis system 100 that includes an x-ray tube 110, upper compression paddle 130 and lower breast support platform 135, an anti-scatter grid 160, and a detector 140. The x-ray tube 110 includes a cathode 112, an anode 114 that is mounted on a shaft 116 and rotated by a motor 118, and a tube port 120. Also shown attached to the x-ray tube is a filter 122.

The illustrated x-ray tube may be a glass vacuum tube. Within the cathode 112 is a heated filament. When the x-ray tube is turned ‘on,’ a current is passed through the filament, thereby heating the filament and causing high energy electrons to be dislodged from the filament. A high voltage between cathode and anode causes the electrons to accelerate toward a target location 125 on the anode. The anode is made, for example, from tungsten and is rotated by motor 118 to avoid local overheating of the target location 125 on the anode. Electrons are focused to a specific target location by means of a focusing cup (not shown) that is a separate control electrode that is cylindrical in shape and attached to the cathode, partially surrounding a filament of the cathode. The dislodged electrons collide with the tungsten atoms of the anode and x-ray photons are generated having bremsstrahlung radiation and characteristic line emission spectra. The x-ray photons are emitted in all directions from the target location 125.

The x-ray photons that come out of the tube port 120 are used for imaging. For the purposes of this application, the x-ray photons that come out of the tube port define a static focal spot 127. The static focal spot 127 is the focal spot as it appears from directly beneath the x-ray tube from the perspective of the breast, at or near the chest wall position of the patient. Focal spot characteristics are defined by International Standard CEI IEC 60336. Generally, the focal spot is rectangular in shape and stated for two normal directions of evaluation referred to as the length and width direction. The length direction is generally parallel to a longitudinal axis of the x-ray system, and the width direction is generally perpendicular to the longitudinal axis. The longitudinal axis of an exemplary tomosynthesis system is shown in FIG. 1.

Static focal spot size refers to the focal spot size at any given instantaneous moment in time, as compared to the time-averaged focal spot size during an x-ray exposure of finite time period that is generally referred to herein as the effective focal spot size of an x-ray exposure. The size of the static focal spot 127 significantly affects the heat loading capacity of the x-ray tube. Greater heat loading is possible with larger focal spots, thereby allowing a higher tube current (mA) to be safely provided. The size of the focal spot is determined by a combination of factors including the size and shape of the filament and the shape and bias voltage of the focusing cup. The angle of the target surface further defines a focal spot size along the so-called length direction.

The size of the focal spot is an important factor in a diagnostic x-ray tube because it affects the resolution of the radiography system. More particularly, systems having smaller focal spots have better resolution, so reducing static focal spot size is one design goal. For example, mammography systems may be designed to provide a 0.3 mm focal spot for imaging (0.1 mm focal spot for high magnification images). Movement of the x-ray source during image exposure effectively stretches the width of the static focal spot, resulting in an effective focal spot that is wider than the static focal spot and that decreases image sharpness. The size of the effective focal spot is therefore determined by the size of the static focal spot and the motion of the static focal spot during exposure, and the effective focal spot, that may be understood as a dynamic focal spot, is the accumulation of the static focal spot over time.

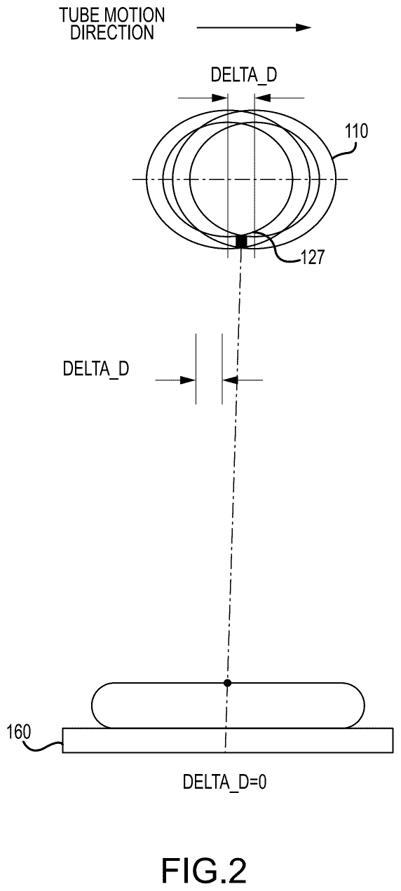

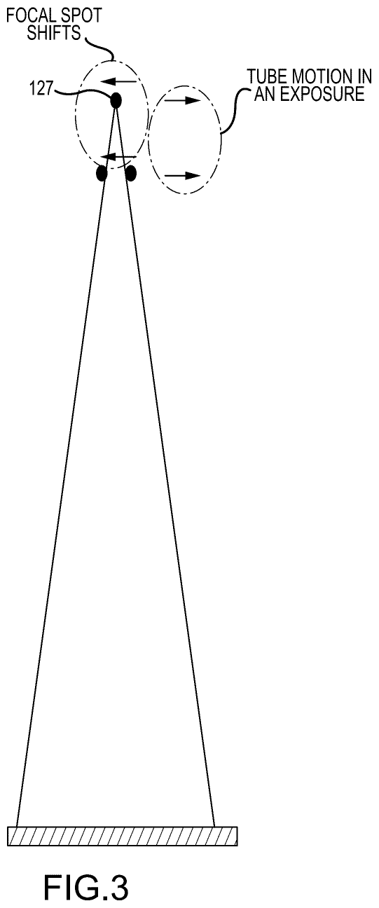

As illustrated in FIGS. 2 and 3, the static focal spot is moved at the same linear speed in a direction opposite to and generally synchronized with the directional movement of the x-ray source during the exposure period. Synchronized movement of the static focal spot and x-ray tube keeps the effective focal spot fixed in space relative to the breast for the entire duration of the exposure and keeps the x-ray field on the detector and breast static.

During a tomosynthesis scan, a series of x-ray pulses are emitted while the x-ray tube moves around the breast. An example x-ray pulse width is 40 ms in each exposure, with a linear scan speed by the x-ray focal spot of about 8 cm per second in examples where the tube moves at a fixed rotational speed around the breast. The focal spot can travel 3.2 mm during each 40 ms x-ray exposure and this is the primary origin of x-ray focal spot motion blur in tomosynthesis, though other sources of blur, such as irregular movement of the tube, also occur. As described above, an MFS x-ray tube allows the focal spot to be deflected for a displacement of 3.2 mm over 40 ms time in the opposite direction of the scan, that can effectively cancel the motion of the focal spot in space and eliminate focal spot motion blur in the image.

For an imaging system with a fixed scan speed, there exists a ramp value that minimizes the effective size of the focal spot at each voltage station and can be predetermined and set to ensure minimal focal spot blurring. The ramp value indicates a control current or voltage applied to the x-ray tube. The displacement of the focal spot is proportional to the applied control current or voltage, which is also referred to herein as the ramp current, the ramp voltage, or, more generally, the ramp value. The MFS ramp calibration of the present disclosure provides a method of finding a ramp value at one or more voltage stations in a MFS system that provides a highest image sharpness or a shortest image blur length. Variations in the tube voltage station impacts image contrast, patient dose, and exposure time.

Large motion blur produces an image that readily appears blurry to human vision. In general, it is easy to reduce the focal spot motion blur length from 3 mm to 0.3 mm by adjusting the control current or voltage. The image will visually turn sharp as the blur length is reduced. In examples, a nominal focal spot size is about 0.3 mm and once the motion blur length approaches and becomes smaller than the focal spot size itself, the image sharpness change may no longer be readily appreciated by a person's vision. This is because the image sharpness will be dominated by the nominal focal spot size instead of the motion blur length. Therefore, a very sensitive method is necessary to assess the image sharpness change while the ramp value is adjusted to determine the optimal value. The method disclosed herein provides for effective assessment of image sharpness even under the extreme condition of zero motion blur length. Additional challenges to ramp value optimization, such as efficient collection of a sufficient number of data points for full system calibration and variations in scan speed, are also addressed by the method of the present disclosure.

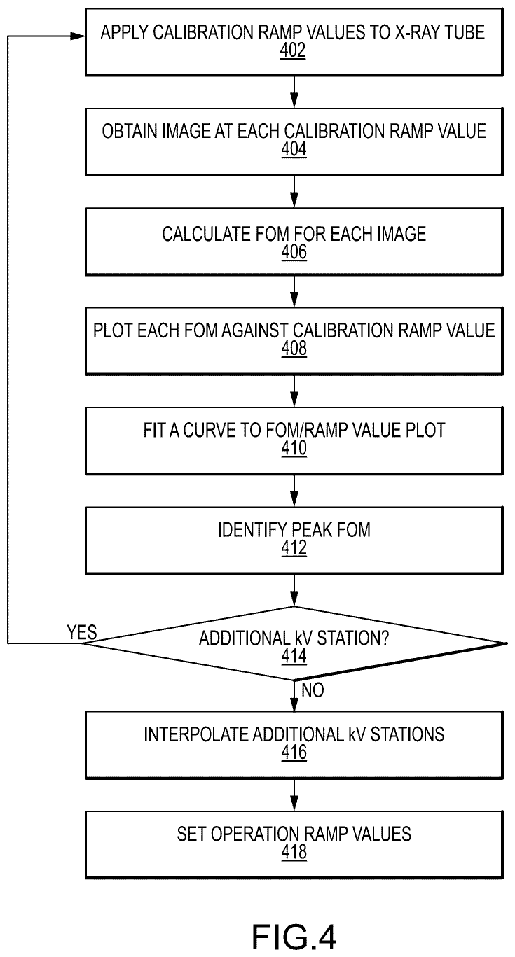

FIG. 4 illustrates an example workflow 400 (400 needs to be labelled in FIG. 4) for calibrating an MFS x-ray tube to optimize a predetermined ramp value. Workflow 400 finds the optimal control parameter (e.g., control current or voltage, referred to as ramp value) that effectively offsets the focal spot travel distance or displacement during each x-ray exposure.

Workflow 400 may be performed as an initial calibration on a new MFS system or as part of quality control and maintenance on a system in operation. Workflow 400 may be an automated process self-executed by the system or may be initiated or controlled by an external system or an operator or technician. Workflow 400 may be executed at one or more voltage stations to facilitate determination of a predetermined ramp value for each voltage station. Workflow 400 may be repeated at different voltage stations until sufficient voltage stations have been evaluated to determine ramp values with a highest measured sharpness for a range of selectable voltages. For example, workflow 400 may be executed at a number of key voltage stations within a range of operational voltage stations to enable determination of a full range of ramp values by interpolation.

At 402, calibration ramp values are applied to the x-ray tube. Since the x-ray tube is configured to take multiple exposures per scan, e.g., 15 exposures per an instance of a tomosynthesis scan, multiple ramp values can be applied and evaluated using one calibration scan. In the case of an exemplary tomosynthesis scan of 15 exposures, 15 ramp values may be evaluated in the course of the calibration scan, with a different ramp value applied at each exposure. In some instances, multiple scans may be performed with each scan having a different ramp value.

At 404, an image is taken at each calibration ramp value. A phantom, or sharpness test object, may be positioned in the image capture field to assist in evaluating image sharpness. The phantom may be an MTF edge phantom, or a small, high contrast object such as a thin wire or a small sphere. The size of the phantom may be determined in relation to the image capture field, e.g., occupying less than 50%, less than 25%, less than 10%, less than 5%, etc. of the image capture field. Use of a phantom increases the sensitivity of the method and enables effective measurement of smaller motion blur lengths than would otherwise be detectable. For example, a phantom such as an MTF edge phantom may enable sufficient sensitivity for evaluation of blur lengths of less than 0.3 mm (the example nominal size of the focal spot).

It may be desirable to use the MTF edge, as the MTF test is a standard quality control test in tomosynthesis and mammography, allowing the calibration to serve a dual purpose as a quality control test as well. In some instances, one or more phantoms may be used to achieve the desired sensitivity. In examples, two phantoms may be used, such as two MTF edge phantoms or phantoms of different types. An MTF edge phantom for two edges (for example a first edge corresponding to where the pectoral side of a breast would be positioned, and a second edge positioned where the nipple side of the breast would be positioned) in the scan direction are calculated and co-plotted. An average MTF value of the two or more edges may be used as the FOM for each ramp value. The phantom may be positioned at a certain height above the detector surface and a magnification mode set according to the height. For example, a low magnification mode may be used for a phantom positioned closer, e.g., 4-8 cm, from the detector or a high magnification mode for a phantom positioned farther, e.g., 40-50 cm, from the detector.

In some instances, a calibration scan may be performed in each of a clockwise and counterclockwise direction. For example, step 404 may be executed twice in sequence, first with the tube moving in a clockwise direction and then with the tube moving in a counterclockwise direction. In such instances, at least two images may be obtained at each ramp value. Calibration scans may be performed in one or more view modes. For example, images taken during a calibration scan may be taken using one or a combination of a cranial-caudal view mode, a left-medial-lateral-oblique view mode, or a right-medial-lateral-oblique view mode.

At 406, a figure of merit (FOM) is calculated for each image. The FOM is used to quantify any motion blur in the image. Motion blur may be the blur length in an image or may be evaluated with a more complex analysis like a width of a line spread function, or the amplitude of a modulation transfer function (MTF), along the scan direction. A line spread function provides a measure of a system's ability to form sharp images and may be determined by measuring a spatial density distribution on film of an x-ray image of a narrow slit. The line spread function measures a relative illuminance distribution in the narrow slit and may be understood as a direct measure of light diffusion in screens and film. MTF is a measure of the ability of the system to image, frequency for frequency, a radiation pattern in that intensity varies sinusoidally with distance in one dimension in the object plane. Thus, both the line spread function and MTF provide parameters that can be used as a measure of the image deterioration due to light diffusion. The line spread function and MTF are mathematically related (Fourier transform pair) so that when one has been measured, the other can be calculated. In one example, MTF amplitudes at a given line pair per millimeter (lp/mm) frequency, may be reported as a figure of merit for comparison.

In examples using two or more edges or phantoms, an FOM, such as an MTF value, may be calculated for each edge or phantom used. An average of two or more FOM values calculated may be used in examples using multiple phantoms or multiple scans, or both. For example, in instances including both a clockwise and counterclockwise calibration scan, the FOM is calculated for each image. In such instances, at least two FOMs may be determined for each ramp value. Depending on the system, substantially similar, or even identical, FOMs may be determined in both the clockwise and counterclockwise scan direction for a given ramp value. In some cases, differences between the FOM values for a given ramp value in the clockwise and counterclockwise direction may occur. An average of the two FOMs may be used as the FOM for the given ramp value.

At 408, each FOM is evaluated with the associated ramp value for each image. Evaluating the FOMs may be performed by plotting each FOM against the associated ramp value to establish a curve. In examples, the plot may further account for a default or last calibrated ramp value. In instances, evaluating ramp values in each of the clockwise and counterclockwise directions, FOMs for each direction may be plotted or the average may be plotted.

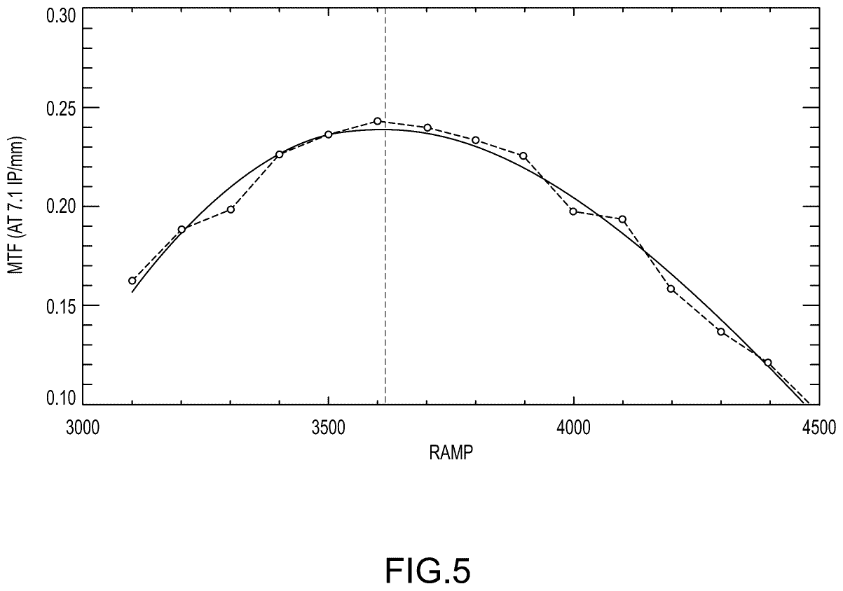

At 410, additional FOM/ramp value points are determined by curve fitting. For example, the curve fitting may be a third order or higher order polynomial curve fitting. FIG. 5 shows an example plot of FOM to ramp value, using MTF for the FOM. The ramp value may be associated with an applied current or an applied voltage. The graph of FIG. 5 also demonstrates the curve fitting to the plot of FOM to ramp value.

At 412, a peak FOM is identified. The peak FOM may be associated with an image having the lowest blur. The peak FOM may be associated with a highest MTF value which indicates a highest image sharpness. The peak FOM may be associated with an average, such as average MTF for two or more phantom edges or an average for two or more exposures at a particular ramp value, e.g., a clockwise exposure and a counterclockwise exposure.

At 414, an assessment determining whether sufficient voltage stations have been evaluated is performed. The calibration scan may be performed at multiple voltage stations. The voltage station chosen for a particular imaging scan may depend on characteristics of the patient and the tissue to be imaged, and the ramp value that produces the image with the highest sharpness or shortest blur may vary with the voltage stations. Therefore, determination of the ramp value to be set as a predetermined ramp value may be different for each voltage station for effective operation of an MFS tube.

In some examples, a ramp value to be set as a predetermined ramp value for each voltage station may be evaluated with a dedicated calibration scan at that voltage station. In some cases, calibration scans at key voltage stations facilitates efficient determination of predetermined ramp values across a range of voltage stations with less time spent on calibration and less wear on the imaging system due to excessive calibration scans. For example, tomosynthesis scans are typically run at voltages ranging from 20 kV-49 kV. Calibration scans may be run at a number of characteristic voltage stations within the range to determine a full corresponding range of ramp values. For example, four voltage stations, such as 22 kV, 30 kV, 40 kV, and 49 kV, may define a curve or slope that permits determination of a ramp value for the remaining voltage stations within the range. Workflow 400 provides for measuring a ramp value at selected kV stations and generating a ramp value for other kV stations through data fitting and interpolation, therefore increasing the speed and efficiency of the calibration for the whole system.

If the determination is yes at 414, and additional voltage stations should be evaluated to complete the calibration, workflow 400 return to 402 and the calibration scan and measurements are repeated at a different voltage station. If the determination is no at 414, and sufficient voltage stations have been evaluated to complete the calibration, workflow 400 proceeds to 416.

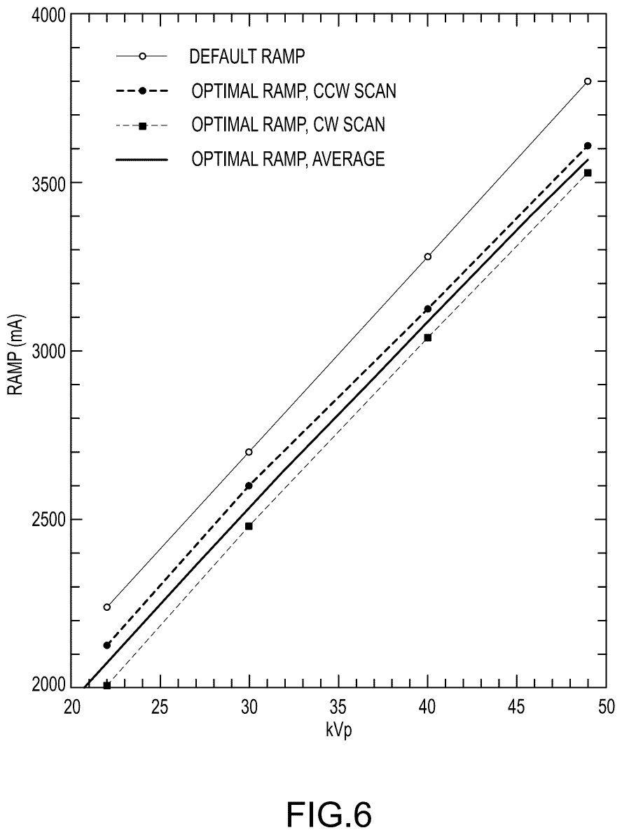

At 416, additional ramp values for unevaluated voltage stations are determined by interpolating a graph of the determined ramp values and voltage stations. Ramp values determined for evaluated voltage stages may be plotted, with ramp values for unevaluated voltage states determined be interpolating curve or slope points. In examples, the graph may be plotted with values from a single scan or both a clockwise and counterclockwise scan, or an average of the two. FIG. 6 depicts an example graph of ramp value to voltage station, using a supplied current as the ramp value.

At 418, the operational ramp values are set for the range of voltage stations. A final set of predetermined ramp values is set for the full range of operational voltages of the imaging system. The ramp values may be automatically set by the system for operational scans based on the outcome of the calibration scans or may be manually set by a technician or operator.

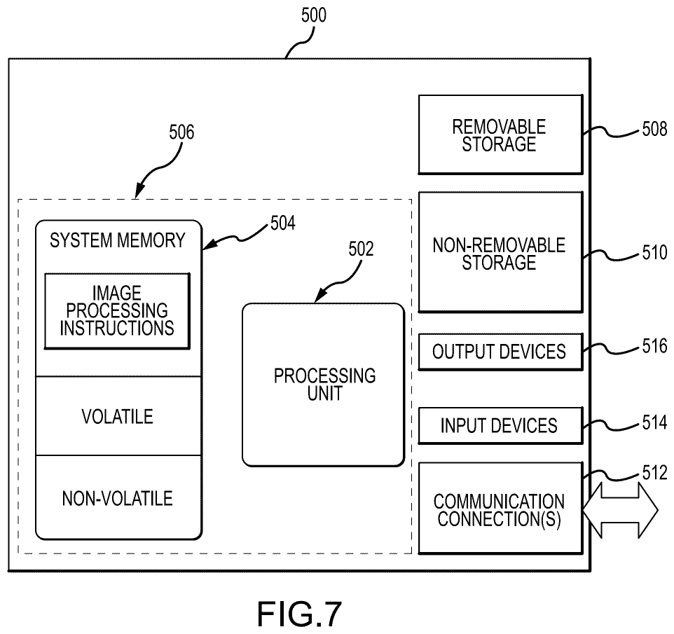

FIG. 7 illustrates one example of a suitable operating environment 500 in that one or more of the present embodiments can be implemented. This operating environment may be incorporated directly into a tomosynthesis system or may be incorporated into a computer system discrete from, but used to control, a tomosynthesis system such as described herein, such as the controller. This is only one example of a suitable operating environment and is not intended to suggest any limitation as to the scope of use or functionality. Other well-known computing systems, environments, and/or configurations that can be suitable for use include, but are not limited to, imaging systems, personal computers, server computers, hand-held or laptop devices, multiprocessor systems, microprocessor-based systems, programmable consumer electronics such as smart phones, network PCs, minicomputers, mainframe computers, tablets, distributed computing environments that include any of the above systems or devices, and the like.

In its most basic configuration, operating environment 500 typically includes at least one processing unit 502 and memory 504. Depending on the exact configuration and type of computing device, memory 504 (storing, among other things, instructions to perform the moving focal spot calibration calculation and methods disclosed herein) can be volatile (such as RAM), non-volatile (such as ROM, flash memory, etc.), or some combination of the two. This most basic configuration is illustrated in FIG. 7 by dashed line 506. Further, environment 500 can also include storage devices (removable, 508, and/or non-removable, 510) including, but not limited to, magnetic or optical disks or tape. Similarly, environment 500 can also have input device(s) 514 such as touch screens, keyboard, mouse, pen, voice input, etc., and/or output device(s) 516 such as a display, speakers, printer, etc. Also included in the environment can be one or more communication connections 512, such as LAN, WAN, point to point, Bluetooth, RF, etc.

Operating environment 500 typically includes at least some form of computer readable media. Computer readable media can be any available media that can be accessed by processing unit 502 or other devices comprising the operating environment. By way of example, and not limitation, computer readable media can comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, solid state storage, or any other tangible medium that can be used to store the desired information. Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The operating environment 500 can be a single computer operating in a networked environment using logical connections to one or more remote computers. The remote computer can be a personal computer, a server, a router, a network PC, a peer device or other common network node, and typically includes many or all of the elements described above as well as others not so mentioned. The logical connections can include any method supported by available communications media. Such networking environments are commonplace in offices, enterprise-wide computer networks, intranets and the Internet.

In some embodiments, the components described herein comprise such modules or instructions executable by computer system 500 that can be stored on computer storage medium and other tangible mediums and transmitted in communication media. Computer storage media includes volatile and non-volatile, removable and non-removable media implemented in any method or technology for storage of information such as computer readable instructions, data structures, program modules, or other data. Combinations of any of the above should also be included within the scope of readable media. In some embodiments, computer system 500 is part of a network that stores data in remote storage media for use by the computer system 500.

The systems and methods disclosed herein may operate on a network. In examples, a device may communicate with one or more servers via a network. Example devices may be a laptop, a personal computer, a smart phone, a PDA, a netbook, or any other type of computing device, such as the computing device in FIG. 7. In embodiments, the servers may also be any type of computing device, such as the computing device illustrated in FIG. 7. The network may be any type of network capable of facilitating communications between a device and one or more servers. For example, the x-rays detected by the detector may be recognized locally in the tomosynthesis system and communicated to another computing device(s) for further processing, such as an image acquisition workstation. Examples of such networks include, but are not limited to, LANs, WANs, cellular networks, and/or the Internet.

In embodiments, the various systems and methods disclosed herein may be performed by one or more server devices. For example, in one embodiment, a single server may be employed to perform the systems and methods disclosed herein, such as the method for calibrating a moving focal spot tube. A device may also perform functionality disclosed herein, such as calibration and image processing, that can then be provided to servers.

In alternate embodiments, the methods and systems disclosed herein may be performed using a distributed computing network, or a cloud network. In such embodiments, the methods and systems disclosed herein may be performed by two or more servers. Although a particular network embodiment is disclosed herein, one of skill in the art will appreciate that the systems and methods disclosed herein may be performed using other types of networks and/or network configurations.

The embodiments described herein can be employed using software, hardware, or a combination of software and hardware to implement and perform the systems and methods disclosed herein. Although specific devices have been recited throughout the disclosure as performing specific functions, one of skill in the art will appreciate that these devices are provided for illustrative purposes, and other devices can be employed to perform the functionality disclosed herein without departing from the scope of the disclosure.

### EXAMPLES

Illustrative examples of the systems and methods described herein are provided below. An embodiment of the system or method described herein may include any one or more, and any combination of, the clauses described below.

Clause 1. A method of determining a predetermined ramp value for an x-ray tube using a sharpness test object positioned between the x-ray tube and an x-ray detector, the method comprising: applying a plurality of ramp values to the x-ray tube; for each applied ramp value, emitting an x-ray energy from the x-ray tube so as to obtain an x-ray image; calculating a figure of merit (FOM) to quantify a motion blur in each x-ray image; and identifying a highest FOM, wherein the highest FOM corresponds to the predetermined ramp value.

Clause 2. The method of clause 1, wherein the ramp value is a current.

Clause 3. The method of clause 1 or 2, wherein the ramp value is a voltage.

Clause 4. The method of any of clauses 1-3, wherein the predetermined ramp value determines a displacement of a focal spot on an anode of the x-ray detector during an x-ray exposure.

Clause 5. The method of any of clauses 1-4, further comprising: plotting each FOM against the ramp value applied on an FOM-ramp value plot; and determining one or more additional data points on the FOM-ramp value plot to obtain an interpolated FOM-ramp value plot.

Clause 6. The method of clause 5, wherein determining one or more additional data points on the FOM-ramp value plot comprises performing a curve fitting.

Clause 7. The method of clause 6, wherein the curve fitting is a polynomial curve fitting.

Clause 8. The method of any of clauses 1-7, wherein calculating the FOM comprises measuring a blur length in each x-ray image.

Clause 9. The method of any of clauses 1-8, wherein calculating the FOM comprises measuring a width of a line spread function for each x-ray image.

Clause 10. The method of any of clauses 1-9, wherein calculating the FOM comprises measuring an amplitude of a modulation transfer function for each x-ray image.

Clause 11. The method of any of clauses 1-10, wherein each of the plurality of ramp values applied comprises a different ramp value.

Clause 12. The method of any of clauses 1-11, wherein the sharpness test object is a modulation transfer function edge phantom.

Clause 13. The method of any of clauses 1-12, wherein the sharpness test object is a wire phantom.

Clause 14. The method of any of clauses 1-13, wherein the sharpness test object is point phantom.

Clause 15. The method of any of clauses 1-14, wherein emitting the x-ray energy is performed during a tomosynthesis scan sequence.

Clause 16. The method of clause 15, wherein tomosynthesis scan is either in a clockwise direction or a counterclockwise direction.

Clause 17. The method of any of clauses 1-16, wherein the method is executed at a first voltage station and repeated at a second voltage station.

Clause 18. The method of clause 17, further comprising plotting a voltage range graph with a first predetermined ramp value with the first voltage station and a second predetermined ramp value with the second voltage station; and interpolating a third predetermined ramp value at a third voltage station based on the voltage range graph.

Clause 19. A method of mitigating focal spot blur in x-ray images by displacing a focal spot relative to motion of an x-ray tube, the method comprising: determining a predetermined ramp current by: applying a plurality of ramp values to the x-ray tube; for each applied ramp value, emitting an x-ray energy from the x-ray tube so as to obtain an x-ray image; calculating a figure of merit (FOM) to quantify a motion blur in each x-ray image; and identifying a highest FOM, wherein the highest FOM corresponds to the predetermined ramp value; and applying the predetermined ramp current to the x-ray tube during image capture.

Clause 20. A system comprising: a computer-readable memory storing executable instructions; and one or more processors in communication with the computer-readable memory, wherein, when the one or more processors execute the executable instructions, the one or more processors perform: applying a plurality of ramp values to the x-ray tube; for each applied ramp value, emitting an x-ray energy from the x-ray tube so as to obtain an x-ray image; calculating a figure of merit (FOM) to quantify a motion blur in each x-ray image; and identifying a highest FOM, wherein the highest FOM corresponds to the predetermined ramp value.

This disclosure described some examples of the present technology with reference to the accompanying drawings, in which only some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skilled in the art.

Although various embodiments and examples are described herein, those of ordinary skill in the art will understand that many modifications may be made thereto within the scope of the present disclosure. Therefore, the specific structure, acts, or media are disclosed only as illustrative examples. Examples according to the technology may also combine elements or components of those that are disclosed in general but not expressly exemplified in combination, unless otherwise stated herein. Accordingly, it is not intended that the scope of the disclosure in any way be limited by the examples provided.

1. A method of determining a predetermined ramp value for an x-ray tube using a sharpness test object positioned between the x-ray tube and an x-ray detector, the method comprising:
applying a plurality of ramp values to the x-ray tube;
for each applied ramp value, emitting an x-ray energy from the x-ray tube so as to obtain an x-ray image;
calculating a figure of merit (FOM) to quantify a motion blur in each x-ray image; and
identifying a highest FOM, wherein the highest FOM corresponds to the predetermined ramp value.

2. The method of claim 1, wherein the ramp value is a current.

3. The method of claim 1, wherein the ramp value is a voltage.

4. The method of claim 1, wherein the predetermined ramp value determines a displacement of a focal spot on an anode of the x-ray detector during an x-ray exposure.

5. The method of claim 1, further comprising:
plotting each FOM against the ramp value applied on an FOM-ramp value plot; and
determining one or more additional data points on the FOM-ramp value plot to obtain an interpolated FOM-ramp value plot.

6. The method of claim 5, wherein determining one or more additional data points on the FOM-ramp value plot comprises performing a curve fitting.

7. The method of claim 6, wherein the curve fitting is a polynomial curve fitting.

8. The method of claim 1, wherein calculating the FOM comprises measuring a blur length in each x-ray image.

9. The method of claim 1, wherein calculating the FOM comprises measuring a width of a line spread function for each x-ray image.

10. The method of claim 1, wherein calculating the FOM comprises measuring an amplitude of a modulation transfer function for each x-ray image.

11. The method of claim 1, wherein each of the plurality of ramp values applied comprises a different ramp value.

12. The method of claim 1, wherein the sharpness test object is a modulation transfer function edge phantom.

13. The method of claim 1, wherein the sharpness test object is a wire phantom.

14. The method of claim 1, wherein the sharpness test object is point phantom.

15. The method of claim 1, wherein emitting the x-ray energy is performed during a tomosynthesis scan sequence.

16. The method of claim 15, wherein tomosynthesis scan is either in a clockwise direction or a counterclockwise direction.

17. The method of claim 1, wherein the method is executed at a first voltage station and repeated at a second voltage station.

18. The method of claim 17, further comprising plotting a voltage range graph with a first predetermined ramp value with the first voltage station and a second predetermined ramp value with the second voltage station; and
interpolating a third predetermined ramp value at a third voltage station based on the voltage range graph.

19. A method of mitigating focal spot blur in x-ray images by displacing a focal spot relative to motion of an x-ray tube, the method comprising:
determining a predetermined ramp current by:
applying a plurality of ramp values to the x-ray tube;
for each applied ramp value, emitting an x-ray energy from the x-ray tube so as to obtain an x-ray image;
calculating a figure of merit (FOM) to quantify a motion blur in each x-ray image; and
identifying a highest FOM, wherein the highest FOM corresponds to the predetermined ramp value; and
applying the predetermined ramp current to the x-ray tube during image capture.

20. A system comprising:
a computer-readable memory storing executable instructions; and
one or more processors in communication with the computer-readable memory, wherein, when the one or more processors execute the executable instructions, the one or more processors perform:
applying a plurality of ramp values to the x-ray tube;
for each applied ramp value, emitting an x-ray energy from the x-ray tube so as to obtain an x-ray image;
calculating a figure of merit (FOM) to quantify a motion blur in each x-ray image; and
identifying a highest FOM, wherein the highest FOM corresponds to the predetermined ramp value.
